(19) United States
(12) Patent Application Publication
(10) Pub. No.: US 2024/0148710 A1
(43) Pub. Date: May 9, 2024

(54) INHIBITORS OF CYTOCHROME P450 FAMILY 7 SUBFAMILY B MEMBER 1 (CYP7B1) FOR USE IN MOBILIZATION OF HEMATOPOIETIC STEM AND PROGENITOR CELLS

(71) Applicant: University of Connecticut, Farmington, CT (US)

(72) Inventor: Hideyuki Oguro, West Hartford, CT (US)

(21) Appl. No.: 18/491,536

(22) Filed: Oct. 20, 2023

Related U.S. Application Data
(60) Provisional application No. 63/418,726, filed on Oct. 24, 2022.

(51) Int. Cl. A61K 31/444; A61K 31/395; A61K 31/4174; A61K 31/496; A61K 31/506; A61K 31/575; A61K 38/18; A61K 38/19; A61K 38/20; A61K 38/27; A61K 38/45; A61K 45/06; A61P 7/06

(52) U.S. Cl. CPC A61K 31/444; A61K 31/395; A61K 31/4174; A61K 31/496; A61K 31/506; A61K 31/575; A61K 38/1866; A61K 38/1891; A61K 38/19; A61K 38/193; A61K 38/195; A61K 38/196; A61K 38/202; A61K 38/27; A61K 38/45; A61K 45/06; A61P 7/06

(57) ABSTRACT

Disclosed are compounds for use in modulating a mobilization of hematopoietic stem and progenitor cells (HSPCs) or hematopoietic stem cells (HSCs) from the bone marrow to the peripheral blood in a subject; wherein the compound inhibits the activity of cytochrome P450 family 7 subfamily B member 1 (CYP7B1 inhibitor).

### CROSS-REFERENCE TO RELATED APPLICATIONS

The present disclosure claims priority to, and the benefit of, U.S. Provisional Application No. 63/418,726, filed 24 Oct. 2022, titled INHIBITORS OF CYTOCHROME P450 FAMILY 7 SUBFAMILY B MEMBER 1 (CYP7B1) FOR USE IN MOBILIZATION OF HEMATOPOIETIC STEM AND PROGENITOR CELLS, which is incorporated by reference herein in its entirety for all purposes.

### GOVERNMENT SUPPORT

This invention was made with government support under DK125747 awarded by the National Institutes of Health. The government has certain rights in the invention.

### FIELD OF THE DISCLOSURE

The subject disclosure relates to the compounds, pharmaceutical compositions, and methods for enhancing or promoting the mobilization of hematopoietic stem and progenitor cells (HSPCs) from the bone marrow to the peripheral blood. The subject disclosure particularly relates to the use of inhibitors of cytochrome p450 family 7 subfamily b member 1 (CYP7B1) enzyme for enhancing or promoting the mobilization of HSPCs from the bone marrow to the peripheral blood. The subject disclosure also relates to compositions including CYPB1 inhibitors, and their uses in enhancing or promoting the mobilization of HSPCs from the bone marrow to the peripheral blood.

### BACKGROUND

Hematopoietic stem cells (HSCs) sustain blood production throughout life and reside primarily in the bone marrow. Mobilization of HSCs from the bone marrow is widely used to collect HSCs in the peripheral blood for clinical transplantation to treat blood disorders, but the standard protocol often fails to mobilize HSCs. Mobilization of hematopoietic stem cells to harvest them in the peripheral blood used for transplantation is mainly performed by the treatment with granulocyte colony-stimulating factor (G-CSF, also called filgrastim or NEUPOGEN®). When filgrastim alone cannot efficiently mobilize hematopoietic stem cells in some patients and donors, a combination of AMD3100 (also called plerixafor or MOZOBIL®) or motixafortide (also called APHEXDA™) with G-CSF is used to induce mobilization of HSCs. However, G-CSF can cause severe sickle cell crises for sickle cell anemia patients and AMD3100 can harm the fetus when administered to pregnant women. Thus, alternative HSC mobilization strategies are needed to improve patient outcomes.

### SUMMARY

In an aspect, disclosed is a compound for use in modulating a mobilization of hematopoietic stem and progenitor cells (HSPCs) or hematopoietic stem cells (HSCs) in a subject, wherein the compound inhibits the activity of cytochrome P450 family 7 subfamily B member 1 (CYP7B1 inhibitor).

In an additional aspect, disclosed is a pharmaceutical composition including therapeutically effective amount of at least one of the compounds disclosed herein.

In a further aspect, disclosed is a method of modulating a mobilization of hematopoietic stem and progenitor cells (HSPCs) or hematopoietic stem cells (HSCs) in a subject in need thereof, the method including: optionally providing, and administering to the subject a therapeutically effective amount of at least one of the compounds disclosed herein, and/or the composition disclosed herein.

In any aspect or embodiment described herein, the compound has the following formula (I):

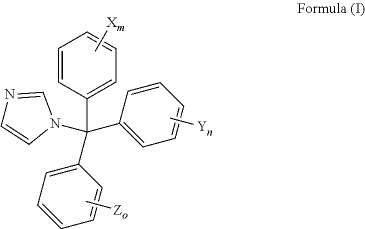

wherein in Formula (I),
each X, Y, and Z is independently selected from the group consisting of (C₁-C₄)alkyl and an electronegative substituent (e.g., each electronegative substituent is independently selected from the group consisting of F, Cl, Br, I, NO₂, CF₃, CN, SCH₃, and OCH₃); and
m, n, and o are each independently 0, 1, 2, 3, 4, or 5;
or a pharmaceutically acceptable salt, solvate or hydrate thereof.

These and other aspects of the present disclosure are described in more detail below.

### BRIEF DESCRIPTION OF THE DRAWINGS

The accompanying drawings, which are incorporated into and form a part of the specification, illustrate several embodiments of the present disclosure and, together with the description, serve to explain the principles of the disclosure. The drawings are only for the purpose of illustrating embodiments of the disclosure, are not necessarily drawn to scale, and are not to be construed as limiting the disclosure. Further objects, features, and advantages of the disclosure will become apparent from the following detailed description taken in conjunction with the accompanying figures showing illustrative embodiments of the disclosure.

FIG. 1 shows the experiment design to study synergistic effects of 27-hydroxycholesterol (27HC) and mobilizing agents on mouse HSPC mobilization.

FIG. 2A shows the number of colony-forming units of HSPCs (CFU-C which represent HSPCs) in the peripheral blood of mice after the indicated treatments (n=4). FIG. 2B shows the number of CFU-granulocyte, macrophage, erythroblast, and megakaryocyte (CFU-gmEM, which represents the most primitive subset of HSPCs that retain multipotency) in the peripheral blood of mice treated with the indicated agents (n=4). FIGS. 2C and 2D show the number of CFU-C (FIG. 2C) or CFU-gmEM (FIG. 2D) in the peripheral blood of mice after the indicated treatments (n=4). Mice were subcutaneously treated with 27HC and/or G-CSF for 4 days, and then treated with AMD3100 one hour before analyses, or vehicle control for each of the above. Data represent mean±S.D. *P<0.05, **P<0.01, ***P<0.001 by one-way ANOVA.

FIGS. 3A and 3B show the number of CFU-C (FIG. 3A) or CFU-gmEM (FIG. 3B) in the peripheral blood of mice after the indicated treatments (n=3). FIGS. 3C and 3D show the number of CFU-C (FIG. 3C) or CFU-gmEM (FIG. 3D) in the peripheral blood of mice after the indicated treatments (n=4). Mice were treated with clotrimazole intraperitoneally for 1 hour (1 hr), 1 day (1 d), 2 days (2 d), or 4 days (4 d) (FIGS. 3A and 3B) or 4 days (FIGS. 3C and 3D) and/or G-CSF subcutaneously for 4 days, and then treated with AMD3100 subcutaneously one hour before analyses. Data represent mean±S.D. *P<0.05, **P<0.01, ***P<0.001 by one-way ANOVA.

FIG. 4A shows the number of CFU-C in the peripheral blood of mice carrying homozygous human sickle cell hemoglobin beta alleles (HBB S/S) or wildtype human hemoglobin beta alleles (HBB A/A) after the indicated treatments (n=4). FIG. 4B shows the number of CFU-gmEM in the peripheral blood of mice after the indicated treatments (n=4). Mice were treated with clotrimazole intraperitoneally for 4 days and then treated with AMD3100 (AMD) subcutaneously one hour before analyses. Data represent mean±S.D. **P<0.01, ***P<0.001 by one-way ANOVA.

FIG. 5A shows the number of CFU-C in the peripheral blood of mice after the indicated treatments (n=4). FIG. 5B shows the number of CFU-gmEM in the peripheral blood of mice after the indicated treatments (n=4). Mice were treated with clotrimazole, ketoconazole, voriconazole, metyrapone, or tioconazole via oral gavage for 4 days and then treated with AMD3100 (AMD) subcutaneously one hour before analyses. Data represent mean±S.D. *P<0.05, **P<0.01, ***P<0.001 by one-way ANOVA.

### DETAILED DESCRIPTION

Hematopoietic stem cells (HSCs) are capable of both self-renewal and differentiation to maintain the entire blood system throughout life and are the functional units of bone marrow transplantation. In adults, HSCs reside primarily in the bone marrow and their number is tightly regulated under steady-state conditions. However, acute demands on the hematopoietic system, such as post-transplantation, pregnancy, blood loss, and myeloablation, promote HSC proliferation and mobilization to extramedullary tissues, such as the spleen, to increase production of blood cells. While the mechanisms that regulate HSC numbers and residence in the bone marrow (BM) under steady-state conditions have been extensively characterized, the mechanisms that regulate HSC proliferation and mobilization in response to hematopoietic demands are less well understood.

To better understand how these key HSC behaviors are regulated, pregnancy in mice has been used as a model system. It was previously found that HSC proliferation, mobilization, and extramedullary hematopoiesis are induced during pregnancy, when maternal blood volume expands rapidly, to replenish red blood cells. These responses are dependent on HSC expression of estrogen receptor α (ERα). Signaling through this nuclear hormone receptor can be triggered by 17β-estradiol (E2), as well as 27-hydroxycholesterol (27HC), the endogenous ER ligand that is a direct metabolite of cholesterol. 27HC induces a unique conformational change in estrogen receptors (ERs) that is different from E2 and other ER ligands. Different ER-ligand complexes also engage functionally distinct coregulators by selective recruitment of coactivators and corepressors. Thus, different ER ligands have different effects on ER function, leading to differential effects on gene expression.

It was recently demonstrated that the treatment of mice with E2 induces HSC proliferation but not mobilization, whereas treatment with 27HC induces ERα-dependent HSC mobilization but not proliferation. Additionally, mice deficient for the sterol hydroxylase, Cyp27a1, which is a mitochondrial cytochrome P450 necessary to generate 27HC by metabolizing cholesterol, exhibit significantly reduced 27HC levels, as well as reduced HSC mobilization and spleen erythropoiesis during pregnancy. However, the mechanism by which E2 and 27HC, two endogenous ERα ligands, differentially regulate HSCs is unknown.

Mobilization of HSCs into the peripheral blood is widely used in clinical allogeneic and autologous transplantation to treat many blood disorders. However, the HSC supply is not sufficient, and a considerable number of healthy donors and patients fail to mobilize HSCs via the standard mobilization protocol. Thus, improved strategies for inducing HSC mobilization are needed to improve patient outcomes. Based on the surprising and unexpected finding that 27HC induce HSC mobilization, disclosed herein is the use of the upregulation of 27HC-ERα signaling to improve the collection of mobilized HSCs for transplantation. As the role of this signaling pathway in HSC function has not been previously addressed, this is expected to open new avenues by identifying the downstream genes that effect HSC proliferation and mobilization.

Disclosed herein is of the use of 27HC-ERα signaling to improve current HSC-mobilizing methods, and compounds, pharmaceutical compositions, and methods for enhancing or promoting the mobilization of hematopoietic stem and progenitor cells (HSPCs) from the bone marrow to the peripheral blood.

The present inventors demonstrated that HSCs divide more often in female mice, as compared to male mice, prompted the present inventors to investigate the potential role of the estrogen receptors (ERs) in the regulation of HSC behavior. The present inventors found that ERα, but not ERβ, is highly expressed by HSCs, and E2, an endogenous ERα agonist, increases HSC proliferation in both female and male mice. E2 levels increase during pregnancy, when extramedullary hematopoiesis (EMH) is induced to increase the production of red blood cells. In addition to increased HSC proliferation, EMH during pregnancy requires increased HSC mobilization, which the present inventors found to also depend on ERα in HSCs. Further, the present inventors found that administering E2 to male mice and non-pregnant females induces HSC proliferation but not mobilization. Given that ERα activity is also regulated by 27HC, the most abundant oxysterol in the plasma of mice and humans, 27HC induces HSC mobilization (but not proliferation), and mobilization is dependent on ERα in hematopoietic cells. Confirming the importance of 27HC, a direct metabolite of cholesterol generated by the mitochondrial cytochrome P450 sterol hydroxylase, Cyp27a1, Cyp27a1-deficient mice significantly reduces 27HC levels, as well as reduced HSC mobilization and EMH during pregnancy, while HSC proliferation in the BM during pregnancy was not affected. Thus, through their differential effects on HSC behavior in vivo—E2 and 27HC act in concert to promote hematopoiesis during pregnancy.

HSC mobilization is also critical to clinical HSC transplantation to treat blood disorders and malignancies. Donor HSCs are predominantly collected using protocols that mobilize them into the peripheral blood using HSC-mobilizing agents, such as granulocyte-colony stimulating factor (G-CSF). However, as much as 40% of individuals still fail to reach the minimum HSC collection threshold required for transplantation using traditional strategies. Such mobilization failure can increase patient morbidity because patients cannot proceed to transplantation. In addition, G-CSF treatment has severe adverse effects on patients with particular diseases, such as sickle cell disease. Thus, advances in mobilization strategies that could improve the outcomes of HSC collection, without introducing additional side effects, are needed to improve patient outcomes.

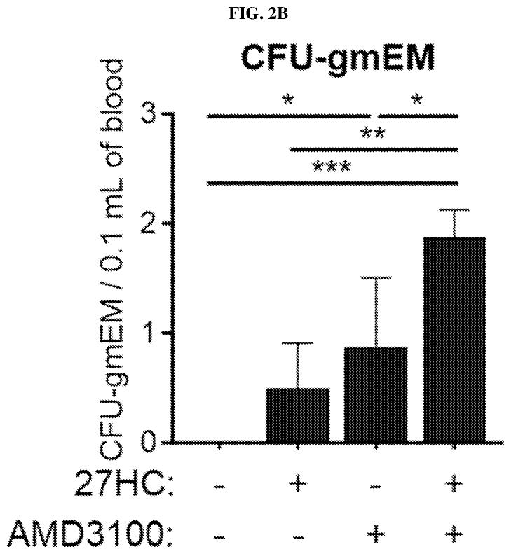

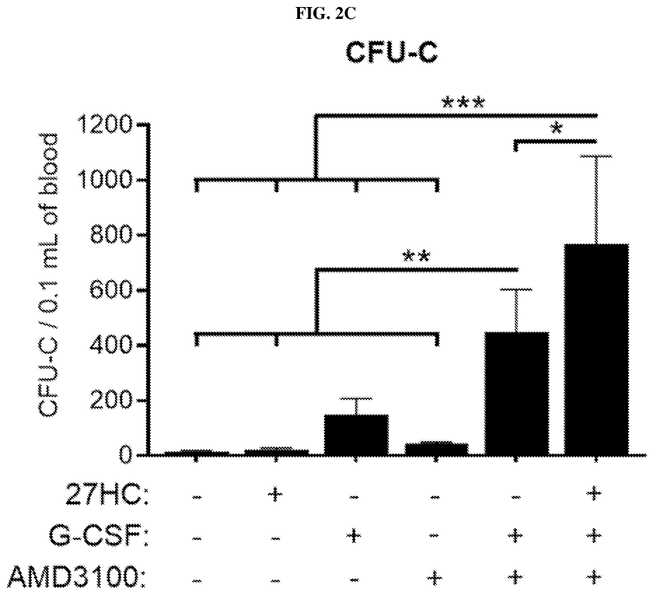

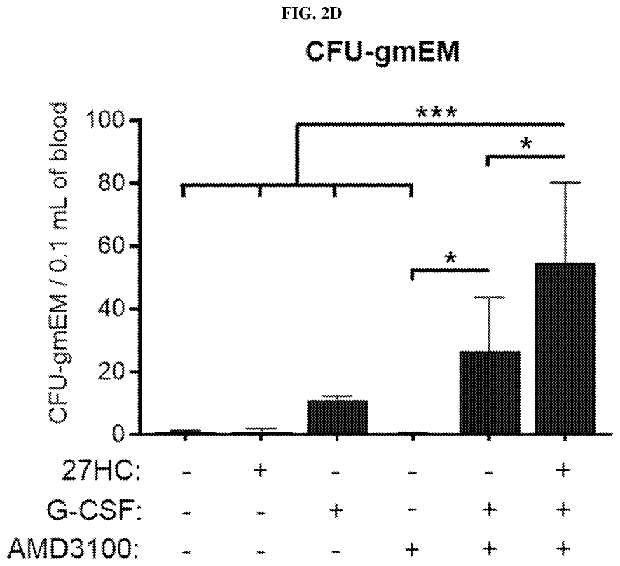

Healthy people and autologous transplantation patients with higher cholesterol levels mobilize a larger number of HSCs naturally or in response to G-CSF treatment, as compared to those with lower cholesterol levels. Similarly, in mice, increased cholesterol levels are known to promote HSC mobilization, while defects in cholesterol efflux pathways are associated with elevated serum G-CSF levels. This latter observation has led to a widely-accepted view that elevated cholesterol levels mobilize HSCs via G-CSF. However, as the data presented herein show, elevated cholesterol levels promote HSC mobilization via increased 27HC production, because treating mice with 27HC promotes HSC mobilization (FIG. 2) and 27HC levels increase as cholesterol levels increase.

Despite their distinct affects on HSC behavior, 27HC and E2 are both ligands for ERα, a well-characterized nuclear receptor transcription factor that regulates cell proliferation and homeostasis in many tissues. ER ligands can differentially affect ER conformation. While not wishing to be bound by theory, it is understood that upregulation of 27HC-ERα signaling can enhance current HSC-mobilizing methods. Disclosed herein are the effect of 27HC treatment using compounds that inhibit the activity of cytochrome P450 family 7 subfamily B member 1 (CYP7B1 inhibitor) in combination with clinically-used HSC-mobilizing agents on mobilization of transplantable HSCs.

To test whether co-administration of 27HC with G-CSF plus another clinically-used HSC mobilizing agent, AMD3100 (a CXCR4 chemokine receptor antagonist that is also known as plerixafor), further enhances HSC mobilization. Two-month-old C57BL/6 mice were treated with a single agent alone, a combination of two agents (27HC+AMD3100, or G-CSF+AMD3100), all three agents combined, or vehicle alone (2-hydroxypropyl-β-cyclodextrin for 27HC, PBS for G-CSF, and PBS for AMD3100). The protocol was as follows, of 27HC (10 mg/kg/day) and/or G-CSF (250 μg/kg/day) were subcutaneously injected daily, for 4 days, and AMD3100 (5 mg/kg) was subcutaneously injected one hour before the peripheral blood was harvested because the peak mobilization of HSCs by AMD3100 injection occurs one hour post administration. The results demonstrated that co-administration of 27HC and AMD3100 mobilized colony-forming HSPCs significantly more than AMD3100 alone (FIGS. 2A and 2B). The surprising and unexpected discovery that co-administration of AMD3100 with G-CSF can rescue HSC mobilization in patients who fail to mobilize by G-CSF alone suggests the clinical potential of 27HC co-administration (FIGS. 2C and 2D). This demonstrates that administration of the combination of three agents mobilized more colony-forming HSPCs as compared to a single agent alone or a combination of two agents (FIGS. 2C and 2D).

In an aspect, disclosed the use of 27-hydroxycholesterol (27HC) in modulating a mobilization of hematopoietic stem and progenitor cells (HSPCs) or hematopoietic stem cells (HSCs) in a subject. In any aspect or embodiment described herein, the 27HC modulates the mobilization of hematopoietic stem and progenitor cells (HSPCs) or hematopoietic stem cells (HSCs) from bone marrow to peripheral blood.

In another aspect, disclosed is a compound for use in modulating a mobilization of hematopoietic stem and progenitor cells (HSPCs) or hematopoietic stem cells (HSCs) in a subject, wherein the compound inhibits the activity of cytochrome P450 family 7 subfamily B member 1 (CYP7B1 inhibitor). In any aspect or embodiment described herein, the compound modulates the mobilization of hematopoietic stem and progenitor cells (HSPCs) or hematopoietic stem cells (HSCs) from bone marrow to peripheral blood.

In any aspect or embodiment described herein, the compound can be 27-hydroxycholesterol (27HC) or a pharmaceutically acceptable salt, solvate or hydrate thereof.

In any aspect or embodiment described herein, the compound can be any CYP7B1 inhibitor. For example, in any aspect or embodiment described herein, the compound is a CYP7B1 inhibitor that includes or is: an azole compound (e.g., imidazole-based compound and triazole-based compound), such as cyproconazole, ketoconazole, bifonazole, miconazole, clotrimazole, voriconazole, ketoconazole, propiconazole, econazole, tebuconazole, tioconazole, and fluconazole; a pyridine derivative (such as metyrapone); or a combination thereof. Thus, in any aspect or embodiment described herein, the compound or CYP7B1 inhibitor includes or is metyrapone, tioconazole, voriconazole, ketoconazole, clotrimazole, a pharmaceutically acceptable salt, solvate or hydrate thereof, or a combination thereof. For example, in any aspect or embodiment described herein, the compound or CYP7B1 inhibitor includes or is metyrapone, voriconazole, ketoconazole, clotrimazole, a pharmaceutically acceptable salt, solvate or hydrate thereof, or a combination thereof. By way of further example, in any aspect or embodiment described herein, the compound or CYP7B1 inhibitor includes or is clotrimazole or a pharmaceutically acceptable salt, solvate or hydrate thereof.

In any aspect or embodiment described herein, the compound has the following formula (I):

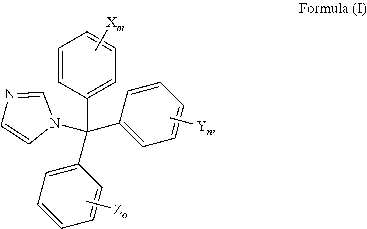

wherein in Formula (I),
each X, Y, and Z is independently selected from the group consisting of (C₁-C₄)alkyl and an electronegative substituent (e.g., each electronegative substituent is independently selected from the group consisting of F, Cl, Br, I, NO₂, CF₃, CN, SCH₃, and OCH₃); and
m, n, and o are each independently 0, 1, 2, 3, 4, or 5;
or a pharmaceutically acceptable salt, solvate or hydrate thereof.

In any aspect or embodiment described herein, the compound modulates the mobilization of hematopoietic stem and progenitor cells (HSPCs) from bone marrow to peripheral blood. In any aspect or embodiment described herein, the compound has the following formula (Ia):

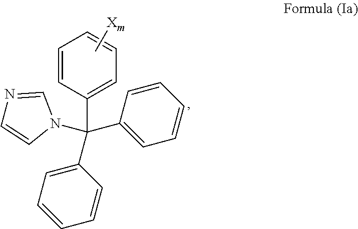

wherein in Formula (Ia),
each X is independently (C₁-C₄)alkyl or an electronegative substituent (e.g., each electronegative substituent is independently selected from the group consisting of F, Cl, Br, I, NO₂, CF₃, CN, SCH₃, and OCH₃); and
m is 0, 1, 2, 3, 4, or 5;
or a pharmaceutically acceptable salt, solvate or hydrate thereof.

In any aspect or embodiment described herein, in Formula (Ia), m is 1 or 2; and each X is independently (C₁-C₄)alkyl, F, Cl, Br, I, or a combination thereof; or a pharmaceutically acceptable salt, solvate or hydrate thereof. In any aspect or embodiment described herein, in Formula (Ia), X is F, Cl, Br, or I; and m is 1; or a pharmaceutically acceptable salt, solvate or hydrate thereof.

In any aspect or embodiment described herein, the compound modulates the mobilization of hematopoietic stem and progenitor cells (HSPCs) from bone marrow to peripheral blood. In any aspect or embodiment described herein, the compound has the following formula (II):

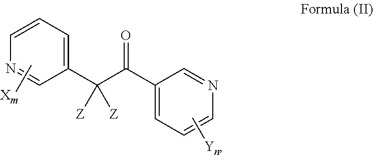

wherein in Formula (II),
each X, Y, and Z is independently selected from the group consisting of (C₁-C₄)alkyl and an electronegative substituent (e.g., each electronegative substituent is independently selected from the group consisting of F, Cl, Br, I, NO₂, CF₃, CN, SCH₃, and OCH₃); and
m and n are each independently 0, 1, 2, 3, 4, or 5;
or a pharmaceutically acceptable salt, solvate or hydrate thereof.

In any aspect or embodiment described herein, the compound modulates the mobilization of hematopoietic stem and progenitor cells (HSPCs) from bone marrow to peripheral blood. In any aspect or embodiment described herein, the compound has the following formula (III):

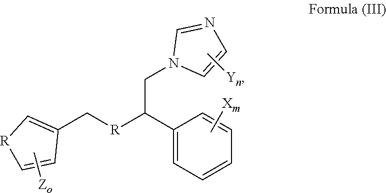

wherein in Formula (III),
each X, Y, and Z is independently selected from the group consisting of (C₁-C₄)alkyl and an electronegative substituent (e.g., each electronegative substituent is independently selected from the group consisting of F, Cl, Br, I, NO₂, CF₃, CN, SCH₃, and OCH₃);
R is S or O; and
m, n, and o are each independently 0, 1, 2, 3, 4, or 5;
or a pharmaceutically acceptable salt, solvate or hydrate thereof.

In any aspect or embodiment described herein, the compound has the following formula (IV):

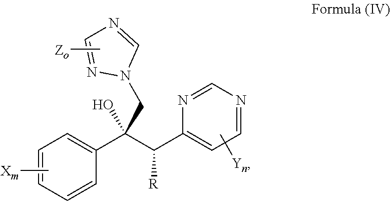

wherein in Formula (IV),
each X, Y, and Z is independently selected from the group consisting of (C₁-C₄)alkyl and an electronegative substituent (e.g., each electronegative substituent is independently selected from the group consisting of F, Cl, Br, I, NO₂, CF₃, CN, SCH₃, and OCH₃);
R is (C₁-C₄)alkyl; and
m, n, and o are each independently 0, 1, 2, 3, 4, or 5;
or a pharmaceutically acceptable salt, solvate or hydrate thereof.

In any aspect or embodiment described herein, each electronegative substituent is independently F, Cl, Br, I, NO₂, CF₃, CN, SCH₃, or OCH₃.

In any aspect or embodiment described herein, the compound is metyrapone, tioconazole, voriconazole, ketoconazole, clotrimazole, a pharmaceutically acceptable salt, solvate or hydrate thereof, or a combination thereof. In any aspect or embodiment described herein, the compound is clotrimazole, or a pharmaceutically acceptable salt, solvate or hydrate thereof. In any aspect or embodiment described herein, the compound is metyrapone, or a pharmaceutically acceptable salt, solvate or hydrate thereof. In any aspect or embodiment described herein, the compound is tioconazole, or a pharmaceutically acceptable salt, solvate or hydrate thereof. In any aspect or embodiment described herein, the compound is voriconazole, or a pharmaceutically acceptable salt, solvate or hydrate thereof. In any aspect or embodiment described herein, the compound is ketoconazole, or a pharmaceutically acceptable salt, solvate or hydrate thereof.

In any aspect or embodiment described herein, the compound is used along with (i) AMD3100 or a pharmaceutically acceptable salt, solvate or hydrate thereof, and/or motixafortide or a pharmaceutically acceptable salt, solvate or hydrate thereof, (ii) G-CSF or a pharmaceutically acceptable salt, solvate or hydrate thereof, or (iii) a combination thereof. In any aspect or embodiment described herein, the compound is used in combination of AMD3100, or a pharmaceutically acceptable salt, solvate or hydrate thereof. In any aspect or embodiment described herein, the compound is used in combination of G-CSF, or a pharmaceutically acceptable salt, solvate or hydrate thereof. In any aspect or embodiment described herein, the compound is used in combination of motixafortide, or a pharmaceutically acceptable salt, solvate or hydrate thereof. In any aspect or embodiment described, the compound is used along with (i) AMD3100 or a pharmaceutically acceptable salt, solvate or hydrate thereof, and/or motixafortide or a pharmaceutically acceptable salt, solvate or hydrate thereof, and (ii) G-CSF or a pharmaceutically acceptable salt, solvate or hydrate thereof. In any aspect or embodiment described herein, the subject is human. In any aspect or embodiment described herein, 27-hydroxycholesterol (27HC) is used along with (i) AMD3100 or a pharmaceutically acceptable salt, solvate or hydrate thereof, and/or motixafortide or a pharmaceutically acceptable salt, solvate or hydrate thereof, (ii) G-CSF or a pharmaceutically acceptable salt, solvate or hydrate thereof, or (iii) a combination thereof.

In an additional aspect, disclosed is a pharmaceutical composition including a therapeutically effective amount of at least one of the compounds disclosed herein. In an aspect, disclosed is a pharmaceutical composition including a therapeutically effective amount of clotrimazole, or a pharmaceutically acceptable salt, solvate or hydrate thereof. In any aspect or embodiment described herein, the composition further includes a carrier that includes at least one pharmaceutically acceptable excipient.

In another aspect, disclosed herein is a combination therapy (e.g., a kit) including: (i) a therapeutically effective amount of: (a) at least one compound that inhibits the activity of cytochrome P450 family 7 subfamily B member 1 (CYP7B1 inhibitor) or a composition comprising therapeutically effective amount of at least one CYP7B1 inhibitor; (b) 27-hydroxycholesterol (27HC) or a pharmaceutically acceptable salt, solvate or hydrate thereof; or (c) a combination thereof; and (ii) an effective or synergistically effective amount of at least one additional mobilization agent, as described herein, wherein the at least one compound and the at least one additional mobilization agent are in the same or different containers.

In yet another aspect, disclosed herein is a composition (e.g., therapeutic composition or pharmaceutical composition)_including: a therapeutically effective amount of: (a) at least one compound that inhibits the activity of cytochrome P450 family 7 subfamily B member 1 (CYP7B1 inhibitor) or a composition comprising therapeutically effective amount of at least one CYP7B1 inhibitor; (b) 27-hydroxycholesterol (27HC) or a pharmaceutically acceptable salt, solvate or hydrate thereof; or (c) a combination thereof; and an effective or synergistically effective amount of at least one additional mobilization agent.

In any aspect or embodiment described herein, (i) the at least one compound includes an azole compound, a compound of formula (I), as described herein, or a combination thereof; (ii) the at least one additional mobilization agent includes a hematopoietic growth factor, acetylcorticotropic hormone, a polyanion, a toxin, an antibody that inhibits the interaction between α4 integrin, a chemokine or analogue thereof, a myelosuppressive chemotherapy agent, or a combination thereof; or (iii) a combination thereof.

In any aspect or embodiment described herein, the at least one additional mobilization agent includes (i) a hematopoietic growth factor (e.g., as described herein), (ii) a chemokine or analogue thereof (e.g., as described herein), or (iii) a combination thereof.

In any aspect or embodiment described herein, (i) the hematopoietic growth factor includes granulocyte colony-stimulating factor (G-CSF, pegylated G-CSF, KIT ligand, Interleukin-3, granulocyte-macrophage colony-stimulating factor (GM-CSF), feline McDonough sarcoma like tyrosine kinase 3 (Flt-3) ligand, thrombopoietin, growth hormone, erythropoietin, vascular endothelial cell growth hormone (VEGF), angiopoietin-1, or a combination thereof; (ii) the chemokine or analogue thereof includes chemokine (C-C motif) ligand 3 (CCL3), chemokine (C-X-C motif) ligand 2 (CXCL2 or Groβ), chemokine (C-X-C motif) ligand 8 (CXCL8 or Interleukin-8), C-X-C motif chemokine 12 (CXCL12 or stromal cell-derived factor 1), C-X-C chemokine receptor type 4 (CXCR4) antagonist (such as AMD3100 or a pharmaceutically acceptable salt, solvate or hydrate thereof, motixafortide or a pharmaceutically acceptable salt, solvate or hydrate thereof, or a combination thereof), or a combination thereof; or (iii) a combination thereof.

In any aspect or embodiment described herein, (i) the hematopoietic growth factor includes granulocyte colony-stimulating factor (G-CSF); (ii) the chemokine or analogue thereof includes (a) AMD3100 or a pharmaceutically acceptable salt, solvate or hydrate thereof, (b) motixafortide or a pharmaceutically acceptable salt, solvate or hydrate thereof, or (c) a combination thereof; or (iii) a combination thereof.

In a further aspect, disclosed is a method of modulating a mobilization of hematopoietic stem and progenitor cells (HSPCs) or hematopoietic stem cells (HSCs)in a subject in need thereof, the method including: optionally providing, and administering to the subject a therapeutically effective amount of at least one (e.g., 1, 2, 3, 4, or 5) of the compounds disclosed herein and/or a therapeutically effective amount of the composition disclosed herein. For example, in an aspect, disclosed is a method of modulating a mobilization of hematopoietic stem and progenitor cells (HSPCs) or hematopoietic stem cells (HSCs) in a subject in need thereof, the method including: optionally providing, and administering to the subject a therapeutically effective amount of 27-hydroxycholesterol (27HC), or a pharmaceutically acceptable salt, solvate or hydrate thereof, and/or a therapeutically effective amount of the composition comprising 27-hydroxycholesterol (27HC), or a pharmaceutically acceptable salt, solvate or hydrate thereof. In a further aspect, disclosed is a method of modulating a mobilization of hematopoietic stem and progenitor cells (HSPCs) or hematopoietic stem cells (HSCs) in a subject in need thereof, the method including: optionally providing, and administering to the subject a therapeutically effective amount of 27-hydroxycholesterol (27HC), and/or a therapeutically effective amount of the composition comprising 27-hydroxycholesterol (27HC).

In another aspect, disclosed is a method of modulating a mobilization of hematopoietic stem and progenitor cells (HSPCs) or hematopoietic stem cells (HSCs)in a subject in need thereof, the method including: optionally providing, and administering to the subject a therapeutically effective amount of clotrimazole, or a pharmaceutically acceptable salt, solvate or hydrate thereof, or a pharmaceutical composition including the same. In any aspect or embodiment described herein, the compound is clotrimazole or a pharmaceutically acceptable salt, solvate or hydrate thereof (e.g., clotrimazole), the subject is a human, or a combination thereof.

In any aspect or embodiment described herein, the method modulates the mobilization of hematopoietic stem and progenitor cells (HSPCs) or hematopoietic stem cells (HSCs) from bone marrow to peripheral blood. In any aspect or embodiment described herein, the at least one of the compounds disclosed herein and/or the composition disclosure herein is administered as a monotherapy.

In any aspect or embodiment described here, the at least one of the compounds disclosed herein and/or the composition disclosure herein is administered as a part of combination therapy. In any aspect or embodiment described herein, the method further includes administering to the subject in need thereof at least one additional therapeutic agent (e.g., an additional mobilization agent). In any aspect or embodiment described herein, the method further includes: optionally providing, and administering to the subject in need thereof: (i) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof; (ii) a therapeutically effective amount of G-CSF or a pharmaceutically acceptable salt, solvate or hydrate thereof; or (iii) a combination thereof. In any aspect or embodiment described herein, the method further includes: optionally providing, and administering to the subject (i) a therapeutically effective amount of AMD3100, or a pharmaceutically acceptable salt, solvate or hydrate thereof, (ii) a therapeutically effective amount of motixafortide or a pharmaceutically acceptable salt, solvate or hydrate thereof, or (iii) a combination thereof. In any aspect or embodiment described herein, the method further includes: optionally providing, and administering to the subject a therapeutically effective amount of G-CSF, or a pharmaceutically acceptable salt, solvate or hydrate thereof. In any aspect or embodiment described herein, the method further includes: optionally providing, and administering to the subject (i) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof, and (ii) a therapeutically effective amount of G-CSF, or a pharmaceutically acceptable salt, solvate or hydrate thereof. In any aspect or embodiment described herein, the subject is human.

In any aspect or embodiment described herein, the method further includes administering to the subject at least one additional mobilization agent.

In any aspect or embodiment described herein, the at least one additional mobilization agent includes a hematopoietic growth factor, acetylcorticotropic hormone, a polyanion, a toxin, an antibody that inhibits the interaction between α4 integrin, a chemokine or analogue thereof, a myelosuppressive chemotherapy agent, or a combination thereof.

In any aspect or embodiment described herein, the at least one additional mobilization agent includes a hematopoietic growth factor, a chemokine or analogue thereof, or a combination thereof.

In any aspect or embodiment described herein, the hematopoietic growth factor includes granulocyte colony-stimulating factor (G-CSF, pegylated G-CSF, KIT ligand, Interleukin-3, granulocyte-macrophage colony-stimulating factor (GM-CSF), feline McDonough sarcoma like tyrosine kinase 3 (Flt-3) ligand, thrombopoietin, growth hormone, erythropoietin, vascular endothelial cell growth hormone (VEGF), angiopoietin-1, or a combination thereof.

In any aspect or embodiment described herein, the polyanion includes fucoidans, dextran suphate, polymethacrylic acid, defibrotide, or a combination thereof.

In any aspect or embodiment described herein, the toxin includes pertussis toxin, bacterial toxin, or a combination thereof.

In any aspect or embodiment described herein, the antibody that inhibits the interaction between α4 integerin includes anti-CD49d, anti-VCAM-1, or a combination thereof.

In any aspect or embodiment described herein, the chemokine or analogue thereof includes chemokine (C-C motif) ligand 3 (CCL3), chemokine (C-X-C motif) ligand 2 (CXCL2 or Groβ), chemokine (C-X-C motif) ligand 8 (CXCL8 or Interleukin-8), C-X-C motif chemokine 12 (CXCL12 or stromal cell-derived factor 1), C-X-C chemokine receptor type 4 (CXCR4) antagonist (such as (a) AMD3100 or a pharmaceutically acceptable salt, solvate or hydrate thereof, or (b) motixafortide or a pharmaceutically acceptable salt, solvate or hydrate thereof, or (c) a combination thereof), or a combination thereof.

In any aspect or embodiment described herein, the myelosuppressive chemotherapy agent includes or is cyclophosphamide (Cy), 5′-fluorouracil (5-FU), or a combination thereof.

In any aspect or embodiment described herein, the additional mobilization agent includes or is (i) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof, (ii) a therapeutically effective amount of granulocyte colony-stimulating factor (G-CSF) or a pharmaceutically acceptable salt, solvate or hydrate thereof, or (iii) a combination thereof.

In any aspect or embodiment described herein, the at least one additional mobilization agent includes a chemokine or analogue thereof. For example, in any aspect or embodiment described herein, the additional mobilization agent or chemokine or analogues thereof includes or is (i) a therapeutically effective amount of AMD3100, or a pharmaceutically acceptable salt, solvate or hydrate thereof, (ii) motixafortide or a pharmaceutically acceptable salt, solvate or hydrate thereof, or (iii) a combination thereof.

In any aspect or embodiment described herein, the subject has sickle cell anemia, granulocyte colony-stimulating factor (G-CSF) is not administered, or a combination thereof. For example, in any aspect or embodiment described herein, the subject has sickle cell anemia and granulocyte colony-stimulating factor (G-CSF) is not administered.

In any aspect or embodiment described herein, the additional mobilization agent includes or is a therapeutically effective amount of granulocyte colony-stimulating factor (G-CSF), or a pharmaceutically acceptable salt, solvate or hydrate thereof.

In any aspect or embodiment described herein, the additional mobilization agent includes or is (i) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof, and (ii) a therapeutically effective amount of granulocyte colony-stimulating factor (G-CSF), or a pharmaceutically acceptable salt, solvate or hydrate thereof.

In a further aspect, disclosed is a method of modulating a mobilization of hematopoietic stem and progenitor cells (HSPCs) or hematopoietic stem cells (HSCs) in a subject in need thereof, the method including: optionally providing, and administering to the subject: (i) a therapeutically effective amount of clotrimazole or a pharmaceutically acceptable salt, solvate or hydrate thereof; (ii) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof; and (iii) a therapeutically effective amount of G-CSF or a pharmaceutically acceptable salt, solvate or hydrate thereof.

In another aspect, disclosed is a method of modulating a mobilization of hematopoietic stem and progenitor cells (HSPCs) or hematopoietic stem cells (HSCs) in a subject in need thereof (e.g., a sickle cell patient), the method including: optionally providing, and administering to the subject in need thereof (i) a therapeutically effective amount of clotrimazole or a pharmaceutically acceptable salt, solvate or hydrate thereof, and (ii) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof.

In an aspect, disclosed is a method of modulating a mobilization of hematopoietic stem and progenitor cells (HSPCs) or hematopoietic stem cells (HSCs) in a subject in need thereof, the method including: optionally providing, and administering to the subject a therapeutically effective amount of clotrimazole or a pharmaceutically acceptable salt, solvate or hydrate thereof, and a therapeutically effective amount of G-CSF or a pharmaceutically acceptable salt, solvate or hydrate thereof. In any aspect or embodiment described herein, the subject is human.

In any aspect or embodiment described herein, the compounds and compositions of the present disclosure modulate the mobilization of hematopoietic stem cells (HSCs). For example, in any aspect or embodiment described herein, the method of the present disclosure modulates the mobilization of hematopoietic stem cells (HSCs). Thus, in any aspect or embodiment described herein, the method is a method of mobilizing hematopoietic stem cells (HSCs) (e.g., mobilization from bone marrow to peripheral blood).

In any aspect or embodiment described herein, the compounds and compositions of the present disclosure modulate the mobilization of hematopoietic stem and progenitor cells (HSPCs). For example, in any aspect or embodiment described herein, the method of the present disclosure modulates the mobilization of hematopoietic stem and progenitor cells (HSPCs). Thus, in any aspect or embodiment described herein, the method is a method of mobilizing hematopoietic stem and progenitor cells (HSPCs) (e.g., mobilization from bone marrow to peripheral blood).

In any aspect or embodiment described herein, the subject will have hematopoietic stem and progenitor cells (HSPCs) or hematopoietic stem cells (HSCs) collected from the peripheral blood. Thus, for example, in any aspect or embodiment described herein, the method further comprises collecting (e.g., collecting via apheresis) hematopoietic stem and progenitor cells (HSPCs) or hematopoietic stem cells (HSCs) from the peripheral blood of the subject. In any aspect or embodiment described herein, the subject has sickle cell anemia, neutropenia, received chemotherapy (e.g., has neutropenia), an immunodeficiency, adrenoleukodystrophy (ALD), Hurler syndrome, Krabbe disease (Globoid-Cell Leukodystrophy), Metachromatic Leukodystrophy (MLD), severe aplastic anemia, severe Combined Immunodeficiency (SCID), sickle cell disease (SCD), Wiskott-Aldrich syndrome (WAS), or a combination thereof. For example, in any aspect or embodiment described herein, the subject has a cancer, a blood cancer (e.g., acute lymphoblastic leukemia (ALL), acute myeloid leukemia (AML), chronic lymphocytic leukemia (CLL), chronic myelogenous leukemia (CML), Hodgkin's lymphoma, multiple myeloma, myelodysplastic syndromes (MDS), non-Hodgkin lymphoma (NHL), or a combination thereof), or a combination thereof.

In any aspect or embodiment described herein, the subject has sickle cell anemia, and the method including administering (i) a therapeutically effective amount of at least one compound of the present disclosure and (ii) AMD3100 or a pharmaceutically acceptable salt, solvate or hydrate thereof, (b) motixafortide or a pharmaceutically acceptable salt, solvate or hydrate thereof, or (c) a combination thereof. Thus, in an aspect, the disclosure provides a method of threating a subject with sickle cell anemia, the method includes: optionally providing, and administering: (i) a therapeutically effective amount of at least one compound of the present disclosure (e.g., 27-hydroxycholesterol (27HC) or a pharmaceutically acceptable salt, solvate or hydrate thereof, and/or clotrimazole or a pharmaceutically acceptable salt, solvate or hydrate thereof) and (ii) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof.

The pharmaceutical compositions disclosed herein includes a pharmaceutically acceptable salt, in particular, an acid or base addition salt, of the compound as described herein. The disclosed compositions may be prepared in various forms, such as capsules, suppositories, tablets, food/drink and the like. Optionally, the disclosed compositions may include various pharmaceutically acceptable excipients, such as microcrystalline cellulose, mannitol, glucose, defatted milk powder, polyvinylpyrrolidone, starch and combinations thereof.

The therapeutically effective compounds as described herein may, in accordance with the disclosure, be administered in single or divided doses by the oral, parenteral or topical routes. Administration of the active compound may range from continuous (intravenous drip) to several administrations per day (for example, Q.I.D.) and may include administration routes such as oral, topical, parenteral, intramuscular, intravenous, sub-cutaneous, transdermal (which may include a penetration enhancement agent), buccal, sublingual, intranasal, intraocular, intrathecal, vaginal, and suppository administration, among other routes of administration. The term “parenteral” as used herein includes subcutaneous, intravenous, intramuscular, intra-articular, intra-synovial, intrasternal, intrathecal, intrahepatic, intralesional and intracranial injection or infusion techniques. Enteric coated oral tablets may be used to enhance bioavailability of the compounds from an oral route of administration. The most effective dosage form will depend upon the pharmacokinetics of the particular agent chosen, as well as the type, location and severity of disease, condition or symptom, and the health of the patient. Administration of compounds according to the present disclosure as sprays, mists, or aerosols for intra-nasal, intra-tracheal or pulmonary administration may also be used. The present disclosure therefore also is directed to pharmaceutical compositions including an effective amount of compound as described herein or a pharmaceutically acceptable salt thereof, optionally in combination with a pharmaceutically acceptable carrier, additive or excipient. Compounds according to the present disclosure may be administered in immediate release, sustained release, or controlled release forms. Sustained or controlled release forms can be administered orally, but also in suppository and transdermal or other topical forms. Intramuscular injections in liposomal form or in depot formulation may also be used to control or sustain the release of compound at an injection site.

The compositions as described herein may be formulated in a conventional manner using one or more pharmaceutically acceptable carriers/excipients and may also be administered in controlled-release formulations. It should also be understood that a specific dosage and treatment regimen for any particular patient will depend on the judgment of the treating physician, as based upon a variety of factors, including the activity and bioavailability of the specific compound employed, the age, body weight, general health, sex, diet, time of administration, rate of excretion, drug combination, and the severity of the particular disease or condition being treated.

A patient or subject in need of therapy using a compound according to the methods described herein can be treated by administering to the patient (subject) an effective amount of the compound according to the present disclosure, either alone, or in combination with another known therapeutic agent.

In any aspect or embodiment described herein, the compound is conveniently administered in any suitable unit dosage form, including, but not limited to, a dosage form containing less than 1 milligrams (mg), 1 mg to 3000 mg, or 5 mg to 500 mg of active ingredient per unit dosage form. An oral dosage of about 25 mg-250 mg is often convenient.

In any aspect or embodiment described herein, the active ingredient is, e.g., administered to achieve peak plasma concentrations of the active compound of about 0.00001 to about 30 millimole (mM) or about 0.1 to about 30 micromole (μM). This may be achieved, for example, by the intravenous injection of a solution or formulation of the active ingredient, optionally in saline, or an aqueous medium or administered as a bolus of the active ingredient. Oral administration may also be appropriate to generate effective plasma concentrations of active agent.

The concentration of active compound in the drug composition will depend on absorption, distribution, metabolism, and excretion rates of the drug, as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the physician administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The present disclosure is illustrated and further described in more detail with reference to the following non-limiting examples.

### EXAMPLES

### Example 1: Synergistic Effect of 27HC, G-CSF, and AMD3100

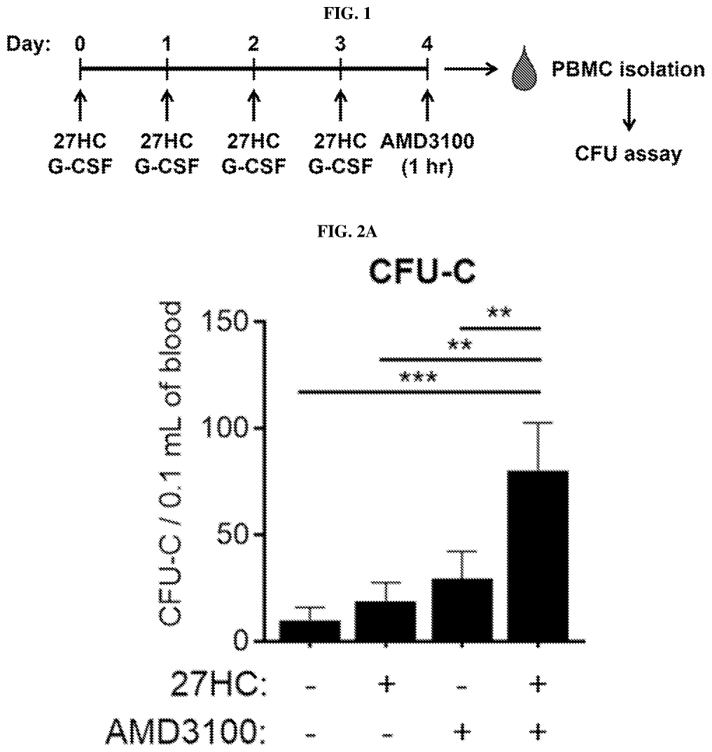

Mice were treated with 27HC (10 mg/kg/day) and/or G-CSF (250 μg/kg/day) for 4 days and then treated with AMD3100 (5 mg/kg) one hour before the peripheral blood collection (FIG. 1). Peripheral blood mononuclear cells (PBMCs) were isolated by density gradient centrifugation, and were seeded in the methylcellulose culture medium. Colonies were counted after 12 days of culture. The results are shown in FIGS. 2A, 2B, 2C and 2D. The two-agent combination treatment with 27HC and AMD3100 generated significantly more CFUs than the AMD3100 single-agent treatment. The three-agent combination treatment with 27HC, G-CSF, and AMD3100 generated significantly more CFUs, including CFU-gmEM, than the two-agent combination treatment with G-CSF and AMD3100. These results demonstrate that the 27HC treatment has synergistic effects with (i) the single-agent AMD3100 treatment or (ii) the two-agent treatment with G-CSF and AMD3100 on mobilizing colony-forming HSPCs.

### Example 2: Synergistic Effect of Clotrimazole, G-CSF, and AMD3100

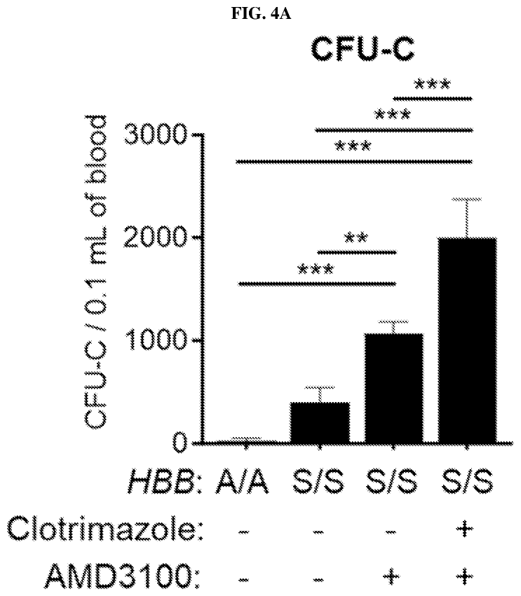

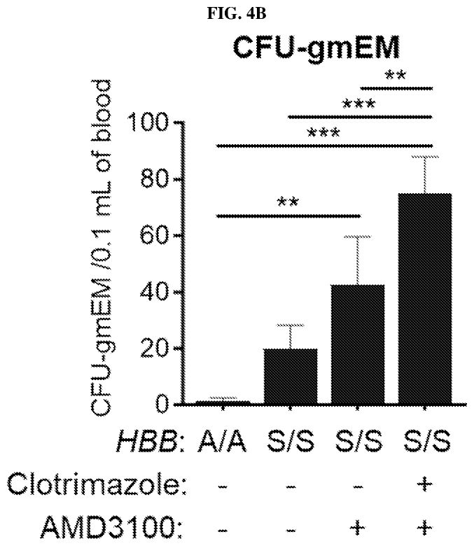

This experiment was performed as described in Example 1, except, clotrimazole (50 mg/kg/day) was used instead of 27HC. The results are shown in FIGS. 4A and 4B. The two-agent combination treatment with clotrimazole and AMD3100 generated significantly more CFUs, including CFU-gmEM, than the single-agent AMD3100 treatment in a sickle cell model mice (Townes model mice, B6; 129-Hbb^{tm2(HBG1,HBB*)Tow} Hba^{tm1(HBA)Tow}). These results demonstrate that the clotrimazole treatment has a synergistic effect with AMD3100 on mobilizing colony-forming HSPCs in sickle cell disease.

### Example 3: Effect of Clotrimazole and AMD3100 on Mobilizing HSPCs in Sickle Cell Model Mice

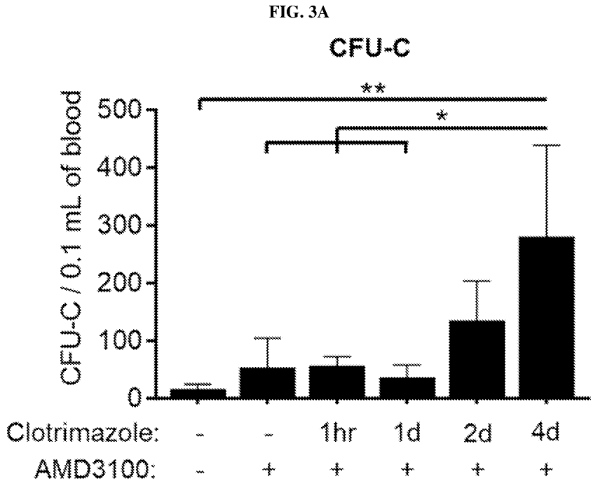

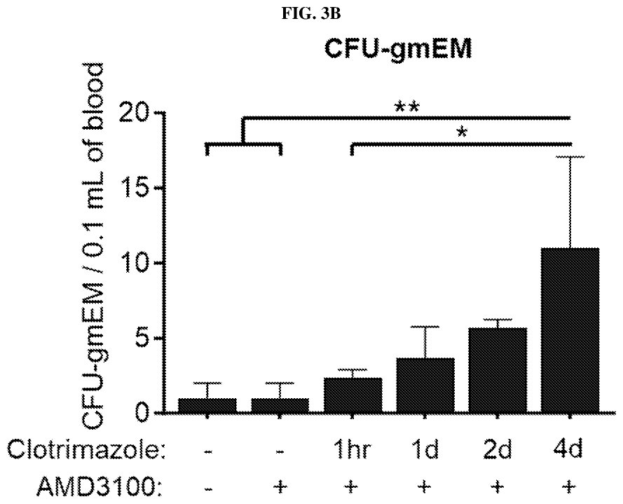

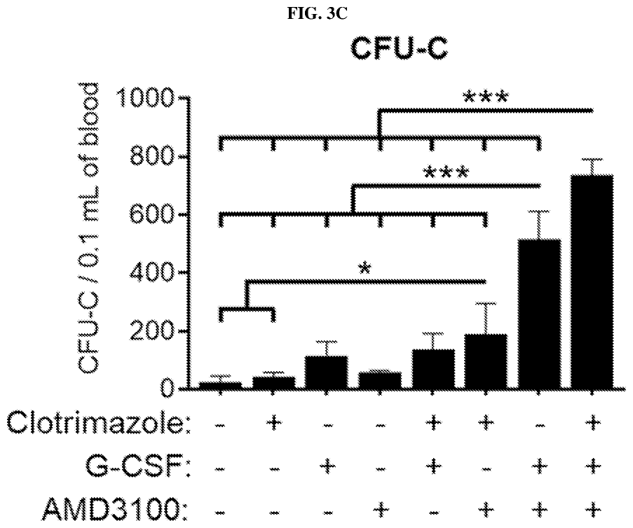

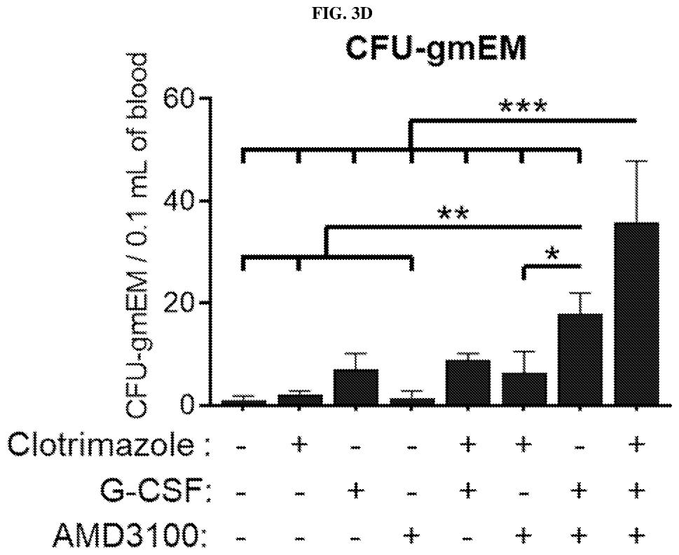

This experiment was performed as described in Example 1, except, clotrimazole (50 mg/kg/day) was used instead of 27HC. The results are shown in FIGS. 3A, 3B, 3C, and 3D. The two-agent combination treatment with clotrimazole and AMD3100 generated significantly more CFUs than the single-agent AMD3100 treatment. The three-agent combination treatment with clotrimazole, G-CSF, and AMD3100 generated more CFUs, including CFU-gmEM, than the two-agent combination treatment with G-CSF and AMD3100. These results demonstrate that the clotrimazole treatment has synergistic effects with (i) the single-agent AMD3100 treatment or (ii) the two-agent treatment with G-CSF and AMD3100 on mobilizing colony-forming HSPCs.

### Example 4: Synergistic Effect of AMD3100 and Ketoconazole, Voriconazole, or Metyrapone

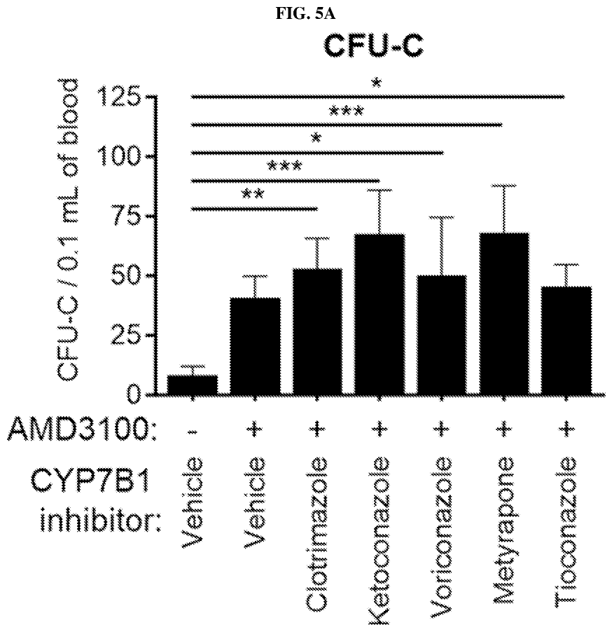

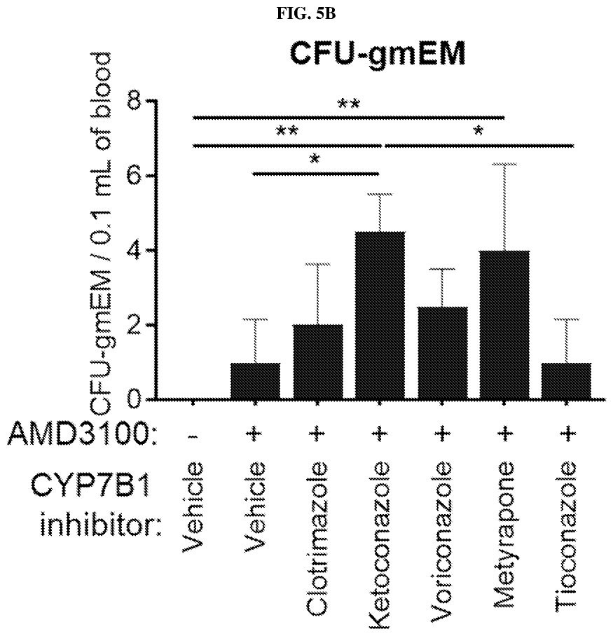

The experiment was performed as described in Example 1, except, oral gavage treatments with clotrimazole (50 mg/kg/day), ketoconazole (50 mg/kg/day), voriconazole (50 mg/kg/day), metyrapone (50 mg/kg/day), or tioconazole (50 mg/kg/day) was used instead of 27HC, and G-CSF was not used. The results are shown in FIGS. 5A, and 5B. There were trends demonstrating that the two-agent combination treatments with AMD3100 and ketoconazole, voriconazole, or metyrapone, but not tioconazole, generated more CFUs, including CFU-gmEM, than the single-agent AMD3100 treatment, similar to the two-agent combination treatment with AMD3100 and clotrimazole. These results demonstrate that not only the clotrimazole treatment, but also ketoconazole, voriconazole, or metyrapone treatments have synergistic effects with the single-agent AMD3100 treatment on mobilizing colony-forming HSPCs.

### DEFINITIONS

Compounds and materials are described using standard nomenclature. Unless defined otherwise, all technical and scientific terms used herein have the same meaning as is commonly understood by one of skill in the art to which the present disclosure belongs. The following terms are used to describe the subject matter of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless otherwise noted.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the subject matter of the present disclosure and does not pose a limitation on the scope of the present disclosure unless otherwise claimed. No language in the specification should be construed as indicating any non-claimed element as essential to the practice of the subject matter of the present disclosure as used herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material including the ingredient, wherein the raw material may be described herein to includes less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual (e.g., by way of a physical or the subject) introduction of a composition into or onto (as appropriate) a subject, a host or cell. Any and all methods of introducing the composition into the subject, host or cell are contemplated according to the present disclosure; the method is not dependent on any particular means of introduction and is not to be so construed. Means of introduction are well-known to those skilled in the art, and also are exemplified herein.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject (e.g., a human subject). In any aspect ore embodiment described herein, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. In any aspect or embodiment described herein, the subject in need of such treatment is a mammal (e.g., a human).

The terms “co-administration” and “co-administering” or “combination therapy” refer to both concurrent administration (administration of two or more therapeutic agents at the same time) and time-varied administration (administration of one or more therapeutic agents at a time different from that of the administration of an additional therapeutic agent or agents), as long as the two or more therapeutic agents are present in the patient to some extent (e.g., at effective amounts) at the same time. In any aspect or embodiment described herein, the at least one compound of the present disclosure is co-administered with at least one additional bioactive agent (e.g., at least one additional mobilization agent). In any aspect or embodiment described herein, the co-administration of such compounds and/or compositions results in synergistic activity and/or therapy (e.g., activity for the mobilization of HSPCs or HSCs from the bone marrow to the peripheral blood).

As used herein, the term “effective amount” or “therapeutically effective amount” refers to the amount of a therapy that is sufficient to obtain a desired/intended result, reduce or ameliorate the severity and/or duration of a disease or disorder, or one or more symptoms thereof, inhibit or prevent the advancement of a disease or disorder, cause regression of a disease or disorder, inhibit or prevent the recurrence, development, onset or progression of one or more symptoms associated with a disease or disorder, detect a disease or disorder, or enhance or improve the prophylactic or therapeutic effect(s) of another therapy (e.g., prophylactic or therapeutic agent). An effective amount can require more than one dose.

Effective amounts may vary depending upon the biological effect desired in the individual, condition to be treated, and/or the specific characteristics of the compound(s) and/or composition of the present disclosure and the individual. In this respect, any suitable dose of the compound(s) and/or composition(s) can be administered to the patient (e.g., human), according to the type of disease to be treated. Various general considerations taken into account in determining the “effective amount” are known to those of skill in the art and are described, e.g., in Gilman et al., eds., Goodman And Gilman's: The Pharmacological Bases of Therapeutics, 8th ed., Pergamon Press, 1990; and Remington's Pharmaceutical Sciences, 17th Ed., Mack Publishing Co., Easton, Pa., 1990, each of which is herein incorporated by reference. The dose of the compound(s) and/or composition(s) of the present disclosure desirably includes about 0.1 mg per kilogram (kg) of the body weight of the patient (mg/kg) to about 400 mg/kg (e.g., about 0.75 mg/kg, about 5 mg/kg, about 30 mg/kg, about 75 mg/kg, about 100 mg/kg, about 200 mg/kg, or about 300 mg/kg). In any aspect or embodiment described herein, the dose of the compound(s) and/or composition(s) according to the present disclosure includes about 0.5 mg/kg to about 300 mg/kg (e.g., about 0.75 mg/kg, about 5 mg/kg, about 50 mg/kg, about 100 mg/kg, or about 200 mg/kg), about 10 mg/kg to about 200 mg/kg (e.g., about 25 mg/kg, about 75 mg/kg, or about 150 mg/kg), or about 50 mg/kg to about 100 mg/kg (e.g., about 60 mg/kg, about 70 mg/kg, or about 90 mg/kg).

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In anu aspect or embodiment described herein, the subject is a human, such as a human being treated or assessed for a disease, disorder or condition, a human at risk for a disease, disorder or condition, a human having a disease, disorder or condition, a human in need of the modulation of the mobilization of HSPCs or HSCs, and/or human being treated for a disease, disorder or condition as described herein. In any aspect or embodiment described herein, the subject is about 1, 2, 3, 4, 5, 6, 7, 8, 9, or 10 years of age. In any aspect or embodiment described herein, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, or 95-100, years of age. Values and ranges intermediate to the above recited ranges are also intended to be part of the present disclosure. In addition, ranges of values using a combination of any of the above-recited values as upper and/or lower limits are intended to be included.

All methods described herein can be performed in a suitable order unless otherwise indicated herein or otherwise clearly contradicted by context. The use of any and all examples, or exemplary language (e.g., “such as”), is intended merely to better illustrate the subject matter of the present disclosure and does not pose a limitation on the scope of the disclosure unless otherwise claimed. No language in the specification should be construed as indicating any non-claimed element as essential to the practice of the present disclosure as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

All compounds are understood to include all possible isotopes of atoms occurring in the compounds. Isotopes include those atoms having the same atomic number but different mass numbers and encompass heavy isotopes and radioactive isotopes. By way of general example, and without limitation, isotopes of hydrogen include tritium and deuterium, and isotopes of carbon include ¹¹C, ¹³C, and ¹⁴C. Accordingly, the compounds disclosed herein may include heavy or radioactive isotopes in the structure of the compounds or as substituents attached thereto. Examples of useful heavy or radioactive isotopes include ¹⁸F, ¹⁵N, ¹⁸O, ⁷⁶Br, ¹²⁵I and ¹³¹I.

“Alkyl” includes both branched and straight chain saturated aliphatic hydrocarbon groups, having the specified number of carbon atoms, generally from 1 to about 8 carbon atoms. The term C₁-C₆alkyl as used herein indicates an alkyl group having from 1, 2, 3, 4, 5, or 6 carbon atoms. Other embodiments include alkyl groups having from 1 to 8 carbon atoms, 1 to 4 carbon atoms or 1 or 2 carbon atoms, e.g., C₁-C₈ alkyl, C₁-C₄ alkyl, and C₁-C₂ alkyl. When C₀-Cₙ alkyl is used herein in conjunction with another group, for example, —C₀-C₂alkyl(phenyl), the indicated group, in this case phenyl, is either directly bound by a single covalent bond (C₀alkyl), or attached by an alkyl chain having the specified number of carbon atoms, in this case 1, 2, 3, or 4 carbon atoms. Alkyls can also be attached via other groups such as heteroatoms as in —O-C₀-C₄ alkyl(C₃-C₇cycloalkyl). Examples of alkyl include, but are not limited to, methyl, ethyl, n-propyl, isopropyl, n-butyl, 3-methylbutyl, t-butyl, n-pentyl, and sec-pentyl.

“Halo” or “halogen” means fluoro, chloro, bromo, or iodo.

All forms (for example solvates, optical isomers, enantiomeric forms, tautomers, polymorphs, free compound and salts) of the compounds of the present disclosure may be employed either alone or in combination with other therapeutic agents, each optionally in a composition that includes at least one carrier and/or excipient.

The composition according to the present disclosure may be administered to a patient by various routes. Examples of routes of administration include, but are not limited to, parenteral, e.g., intravenous, intradermal, subcutaneous, oral, intranasal (e.g., inhalation), transdermal (e.g., topical), transmucosal, and rectal administration. In any aspect or embodiment described herein, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer.

In accordance with any of the aspects or embodiments described herein, the composition according to the present disclosure can be administered orally to a subject in need thereof. Formulations suitable for oral administration can consist of (a) liquid solutions, such as an effective amount of the compound dissolved in diluents (e.g., water, saline, or orange juice) and include an additive, such as cyclodextrin (e.g., α-, β-, or γ-cyclodextrin, hydroxypropyl cyclodextrin) or polyethylene glycol (e.g., PEG400); (b) capsules, sachets, tablets, lozenges, and troches, each containing a predetermined amount of the active ingredient, as solids or granules; (c) powders; (d) suspensions in an appropriate liquid; and (e) suitable emulsions and gels. Liquid formulations may include diluents, such as water and alcohols (for example, ethanol, benzyl alcohol, and the polyethylene alcohols), either with or without the addition of a pharmaceutically acceptable surfactant, suspending agent, or emulsifying agent. Capsule forms can be of the ordinary hard-shelled or soft-shelled gelatin type containing, for example, surfactants, lubricants, and inert fillers (e.g., such as lactose, sucrose, calcium phosphate, and cornstarch). Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such carriers as are known in the art.

The dose administered to the subject (e.g., mammal, such as human and other mammals), in accordance with the present disclosure should be sufficient to affect the desired response (e.g., modulate mobilization of HSPCs and/or HSCs). One skilled in the art will recognize that dosage will depend upon a variety of factors, including the age, condition or disease state, predisposition to disease, genetic defect or defects, and body weight of the mammal. The size of the dose will also be determined by the route, timing and frequency of administration, as well as the existence, nature, and extent of any adverse side-effects that might accompany the administration of a particular composition and the desired effect. It will be appreciated by one of skill in the art that various desired results, conditions, disorders, or disease states may require prolonged treatment involving multiple administrations.

This disclosure describes the best mode or modes of practicing the present disclosure as presently contemplated. This description is not intended to be understood in a limiting sense, but provides an example of the subject matter of the present disclosure solely for illustrative purposes by reference to the accompanying drawings to advise one of ordinary skill in the art of the advantages and construction of the subject matter of the present disclosure.

While the present disclosure describes the subject matter contained herein with reference to exemplary embodiments, it will be understood by those skilled in the art that various changes may be made, and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure is not limited to the particular embodiment or embodiments disclosed as the best mode contemplated for carrying out the disclosed subject matter, but that the disclosure subject matter will include all embodiments falling within the scope of the appended claims. Any combination of the above-described elements in all possible variations thereof is encompassed by the present disclosure unless otherwise indicated herein or otherwise clearly contradicted by context.

### INCORPORATION BY REFERENCE

All U.S. and PCT patent publications and U.S. patents mentioned herein are hereby incorporated by reference in their entirety as if each individual patent publication or patent was specifically and individually indicated to be incorporated by reference. In case of conflict, the present disclosure, including any definitions herein, will control.

### OTHER EMBODIMENTS

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional aspects and embodiments are expressly included within the scope of the present disclosure. The publications and other materials used herein to illuminate the background of the disclosure, and in particular cases, to provide additional details respecting the practice, are incorporated by reference.

Thus, those skilled in the art will recognize, or be able to ascertain using no more than routine experimentation, many equivalents to the specific embodiments of the disclosure described herein. Such equivalents are intended to be encompassed by the following claims. It is understood that the detailed examples and embodiments described herein are given by way of example for illustrative purposes only and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the compounds, compositions, systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A combination therapy comprising:
a therapeutically effective amount of: (a) at least one compound that inhibits the activity of cytochrome P450 family 7 subfamily B member 1 (CYP7B1 inhibitor) or a composition comprising therapeutically effective amount of at least one CYP7B1 inhibitor; (b) 27-hydroxycholesterol (27HC) or a pharmaceutically acceptable salt, solvate or hydrate thereof; or (c) a combination thereof; and
a synergistically effective amount of at least one additional mobilization agent,
wherein the at least one compound and the at least one additional mobilization agent are in the same or different containers.

2. A composition comprising:
a therapeutically effective amount of: (a) at least one compound that inhibits the activity of cytochrome P450 family 7 subfamily B member 1 (CYP7B1 inhibitor) or a composition comprising therapeutically effective amount of at least one CYP7B1 inhibitor; (b) 27-hydroxycholesterol (27HC) or a pharmaceutically acceptable salt, solvate or hydrate thereof; or (c) a combination thereof; and
a synergistically effective amount of at least one additional mobilization agent.

3. The combination therapy of claim 1, wherein:
the at least one compound includes an azole compound, a compound of formula (I), or a combination thereof;
the at least one additional mobilization agent includes a hematopoietic growth factor, acetylcorticotropic hormone, a polyanion, a toxin, an antibody that inhibits the interaction between α4 integrin, a chemokine or analogue thereof, a myelosuppressive chemotherapy agent, or a combination thereof; or
a combination thereof, wherein formula (I) has the chemical structure: 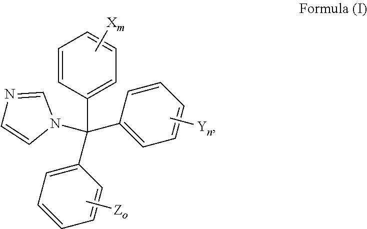
wherein in Formula (I),
each X, Y and Z is independently selected from the group consisting of (C₁-C₄)alkyl and an electronegative substituent; and
m, n, and o are each independently 0, 1, 2, 3, 4, or 5;
or a pharmaceutically acceptable salt, solvate or hydrate thereof.

4. The combination therapy of claim 1, wherein:
the hematopoietic growth factor includes granulocyte colony-stimulating factor (G-CSF, pegylated G-CSF, KIT ligand, Interleukin-3, granulocyte-macrophage colony-stimulating factor (GM-CSF), feline McDonough sarcoma like tyrosine kinase 3 (Flt-3) ligand, thrombopoietin, growth hormone, erythropoietin, vascular endothelial cell growth hormone (VEGF), angiopoietin-1, or a combination thereof;
the chemokine or analogue thereof includes chemokine (C-C motif) ligand 3 (CCL3), chemokine (C-X-C motif) ligand 2 (CXCL2 or Groβ), chemokine (C-X-C motif) ligand 8 (CXCL8 or Interleukin-8), C-X-C motif chemokine 12 (CXCL12 or stromal cell-derived factor 1), C-X-C chemokine receptor type 4 (CXCR4) antagonist, AMD3100 or a pharmaceutically acceptable salt, solvate or hydrate thereof, motixafortide or a pharmaceutically acceptable salt, solvate or hydrate thereof, or a combination thereof; or
a combination thereof.

5. The combination therapy of claim 4, wherein:
the hematopoietic growth factor includes granulocyte colony-stimulating factor (G-CSF);
the chemokine or analogue thereof includes (a) AMD3100 or a pharmaceutically acceptable salt, solvate or hydrate thereof, (b) motixafortide or a pharmaceutically acceptable salt, solvate or hydrate thereof, or (c) a combination thereof; or
a combination thereof.

6. A method of modulating a mobilization of hematopoietic stem and progenitor cells (HSPCs) or hematopoietic stem cells (HSCs) in a subject in need thereof, the method comprising: administering to the subject a therapeutically effective amount of at least one compound that inhibits the activity of cytochrome P450 family 7 subfamily B member 1 (CYP7B1 inhibitor) or a composition comprising therapeutically effective amount of at least one CYP7B1 inhibitor and a carrier that comprises at least one pharmaceutically acceptable excipient.

7. The method of claim 6, wherein the compound modulates the mobilization of hematopoietic stem and progenitor cells (HSPCs) or hematopoietic stem cells (HSCs) from bone marrow to peripheral blood.

8. The method of claim 6, wherein the compound has the following formula (I): 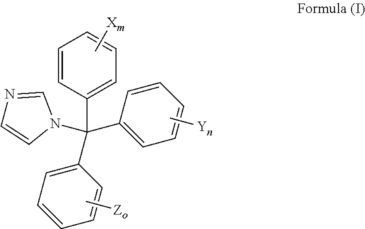
wherein in Formula (I),
each X, Y and Z is independently selected from the group consisting of (C₁-C₄)alkyl and an electronegative substituent; and
m, n, and o are each independently 0, 1, 2, 3, 4, or 5;
or a pharmaceutically acceptable salt, solvate or hydrate thereof.

9. The method of claim 8, wherein each the electronegative substituent is selected from the group consisting of F, Cl, Br, I, NO₂, CF₃, CN, SCH₃ and OCH₃.

10. The method of claim 6, wherein the compound is metyrapone, tioconazole, voriconazole, ketoconazole, clotrimazole, a pharmaceutically acceptable salt, solvate or hydrate thereof, or a combination thereof.

11. The method of claim 6, wherein the compound is clotrimazole, the subject is a human, or a combination thereof.

12. The method of claim 6, wherein the method is administered as part of a combination therapy.

13. The method of claim 6, further comprising:
administering to the subject in need thereof at least one additional therapeutic agent; or
administering to the subject at least one additional mobilization agent.

14. The method of claim 13, wherein the at least one additional mobilization agent includes a hematopoietic growth factor, acetylcorticotropic hormone, a polyanion, a toxin, an antibody that inhibits the interaction between α4 integrin, a chemokine or analogue thereof, a myelosuppressive chemotherapy agent, or a combination thereof.

15. The method of claim 14, wherein:
the hematopoietic growth factor includes granulocyte colony-stimulating factor (G-CSF), pegylated G-CSF, KIT ligand, Interleukin-3, granulocyte-macrophage colony-stimulating factor (GM-CSF), feline McDonough sarcoma like tyrosine kinase 3 (Flt-3) ligand, thrombopoietin, growth hormone, erythropoietin, vascular endothelial cell growth hormone (VEGF), angiopoietin-1, or a combination thereof;
the polyanion includes fucoidans, dextran suphate, polymethacrylic acid, defibrotide, or a combination thereof;
the toxin includes pertussis toxin, bacterial toxin, or a combination thereof;
the antibody that inhibits the interaction between α4 integerin includes anti-CD49d, anti-VCAM-1, or a combination thereof;
the chemokine or analogue thereof includes chemokine (C-C motif) ligand 3 (CCL3), chemokine (C-X-C motif) ligand 2 (CXCL2 or Gro(3), chemokine (C-X-C motif) ligand 8 (CXCL8 or Interleukin-8), C-X-C motif chemokine 12 (CXCL12 or stromal cell-derived factor 1), C-X-C chemokine receptor type 4 (CXCR4) antagonist, AMD3100 or a pharmaceutically acceptable salt, solvate or hydrate thereof, motixafortide or a pharmaceutically acceptable salt, solvate or hydrate thereof, or a combination thereof;
the myelosuppressive chemotherapy agent includes or is cyclophosphamide (Cy), 5′-fluorouracil (5-FU), or a combination thereof; or
a combination thereof.

16. The method of claim 13, wherein the at least one additional mobilization agent includes a hematopoietic growth factor, a chemokine or analogue thereof, or a combination thereof.

17. The method of claim 13, wherein the at least one additional mobilization agent includes:
(i) a therapeutically effective amount of (a) AMD3100 or a pharmaceutically acceptable salt, solvate or hydrate thereof, (b) motixafortide or a pharmaceutically acceptable salt, solvate or hydrate thereof, or (c) a combination thereof;
(ii) a therapeutically effective amount of granulocyte colony-stimulating factor (G-CSF) or a pharmaceutically acceptable salt, solvate or hydrate thereof, or
(iii) a combination thereof.

18. The method of claim 13, wherein the at least one additional mobilization agent includes a chemokine or analogue thereof.

19. The method of claim 18, wherein the chemokine or analogue thereof includes or is (i) a therapeutically effective amount of AMD3100, or a pharmaceutically acceptable salt, solvate or hydrate thereof, (ii) motixafortide or a pharmaceutically acceptable salt, solvate or hydrate thereof, or (iii) a combination thereof.

20. The method of claim 19, wherein:
the subject has sickle cell anemia;
granulocyte colony-stimulating factor (G-CSF) is not administered; or
a combination thereof.
